(19) United States
(12) Patent Application Publication
(10) Pub. No.: US 2009/0081751 A1
(43) Pub. Date: Mar. 26, 2009

(54) NATIVELY GLYCOSYLATED MAMMALIAN BIOLOGICAL MOLECULES PRODUCED BY ELECTROMAGNETICALLY STIMULATING LIVING MAMMALIAN CELLS

(76) Inventors: Thomas J. Goodwin, Kemah, TX (US); Donnie Rudd, Sugar Land, TX (US)

Correspondence Address: LADAS & PARRY LLP, 224 SOUTH MICHIGAN AVENUE, SUITE 1600, CHICAGO, IL, 60604, US

(21) Appl. No.: 12/107,924

(22) Filed: Apr. 23, 2008

Related U.S. Application Data
(62) Division of application No. 11/170,520, filed on Jun. 29, 2005, now abandoned.
(60) Provisional application No. 60/583,976, filed on Jun. 30, 2004.

(51) Int. Cl. C12N 13/00; C07K 1/00

(52) U.S. Cl. 435/173.7; 530/427

(57) ABSTRACT

A composition is disclosed with the composition comprising a mixture of natively glycosylated mammalian biological molecules produced by electromagnetically stimulating living mammalian cells.

### ORIGIN OF THE INVENTION

The invention described herein was made in part by an employee of the United States Government and may be manufactured and used by and for the Government of the United States for governmental purposes without the payment of any royalties thereon or therefor.

### BACKGROUND OF THE INVENTION

1. Field of the Invention

The present invention relates generally to the fields of production of natively glycosylated mammalian biological molecules. Specifically, the present invention relates to a system and process for producing natively glycosylated mammalian biological molecules produced by using electromagnetic fields. More specifically, the present invention relates to a process for producing natively glycosylated mammalian biological molecules by electromagnetically stimulating mammalian cells.

The preferred embodiment utilizes introducing mammalian cells and a carrier medium into a cylindrical chamber and rotating the cylindrical chamber about its axis at a rotational speed sufficient to prevent the cells from substantially contacting the cylindrical walls of the cylindrical chamber and continuing the rotation until the supernatant liquid containing the cells has a significantly increased amount of a mixture of natively glycosylated mammalian biological molecules, and then separating the natively glycosylated mammalian biological molecules into individual molecular entities in significant quantities to be used for therapeutic purposes.

Subjecting the original cell mixture to an electromagnetic field, preferably a time varying electromagnetic field may enhance the process.

2. Description of the Prior Art

In order to more fully understand this invention, a brief discussion of definitions and terms is useful including the following:
Glycosylation: The process of adding sugar units such as in the addition of glycan chains to proteins.
Post-translational modification: The enzymatic processing of a polypeptide chain after translation from messenger RNA and after peptide bond formation has occurred. Examples include glycosylation, acylation, limited proteolysis, phosphorylation, and isoprenylation.
Protein: Any of a group of complex organic compounds which contain carbon, hydrogen, oxygen, nitrogen and usually sulphur, the characteristic element being nitrogen and which are widely distributed in plants and animals. Proteins, the principal constituents of the protoplasm of all cells, are of high molecular weight and consist essentially of combinations of amino acids in peptide linkages. Twenty different amino acids are commonly found in proteins and each protein has a unique, genetically defined amino acid sequence that determines its specific shape and function. They serve as enzymes, structural elements, hormones, immunoglobulins, etc., and are involved in oxygen transport, muscle contraction, electron transport and other activities throughout the body and in photosynthesis.
Polypeptide: A peptide which on hydrolysis yields more than two amino acids, called tripeptides, tetrapeptides, etc., according to the number of amino acids contained.
Peptide: A compound of two or more amino acids where the alpha carboxyl group of one is bound to the alpha amino group of another.
Sulfhydryl: The radical —SH; contained in glutathione, cysteine, coenzyme A, lipoamide (all in the reduced state), and in mercaptans (R-SH).
Myrisolated Proteins: The first proteins to be demonstrated to contain myristic acid were calcineurin B and the catalytic subunit of the cyclic AMP-dependent protein kinase. It was shown that myristic acid (R2) was attached through an amide linkage-amino group of glycine (R1) at the N-terminus of both proteins: to the R1—NH—CO—R2. Wide ranges of proteins of viral and cellular origin are modified by acylation with myristic acid. Myristoylated proteins are localized to the cytosol or to cellular membranes and sometimes to both. Membrane-bound myristoylated proteins interact tightly with the bilayer so that drastic conditions may be used to release them from membranes. It is now well established that myristoylation is able to direct soluble proteins to membranes but the specificity of targeting remains unclear. The function for myristoylation is also not well known. It was speculated that these proteins may represent enzymes involved in lipid metabolism or carrier proteins
Myristic acid: The myristoyl group is one of the less common fatty acyl residues of phospholipids in biological membranes but is found as an N terminal modification of a large number of membrane associated proteins and some cytoplasmic proteins. It is a common modification of viral proteins. In all known examples, the myristoyl residue is attached to the amino group of N terminal glycine. The specificity of the myristoyl transferase enzymes is extremely high with respect to the fatty acyl residue. For many proteins, the addition of the myristoyl group is essential for membrane association. There is some evidence that myristoylated proteins do not interact with free lipid bilayer, but require a specific receptor protein in the target membrane
Granulocyte-colony stimulating factor: A glycoprotein of 25 kD containing internal disulfide bonds. It induces the survival, proliferation, and differentiation of neutrophilic granulocyte precursor cells and functionally activates mature blood neutrophils. Among the family of colony-stimulating factors, G-CSF is the most potent inducer of terminal differentiation to granulocytes and macrophages of leukaemic myeloid cell lines. It is a protein that stimulates the growth and maturation of granulocytes. It is used to promote the recovery of the white cells following chemotherapy. Granulocyte colony stimulating factor (G-CSF) is a glycoprotein that stimulates the survival, proliferation, differentiation and function of neutrophil granulocyte progenitor cells and mature neutrophils. The two forms of recombinant human G-CSF in clinical use (filgrastim and lenograstim) are potent stimulants of neutrophil granulopoiesis and have demonstrated efficacy in preventing infectious complications of some neutropenic states. They can be used to accelerate neutrophil recovery from myelosuppressive treatments. G-CSF decreases the morbidity of cancer chemotherapy by reducing the incidence of febrile neutropenia, the morbidity of high-dose chemotherapy supported by marrow transplantation, and the incidence and duration of infection in patients with severe chronic neutropenia.

Mouse granulocyte colony stimulating factor (G-CSF) was first recognized and purified in Australia in 1983, and groups from Japan and the U.S.A. cloned the human form in 1986. The natural human glycoprotein exists in two forms of 174 and 177 amino acids. The more abundant and more active 174 amino acid form has been used in the development of pharmaceutical products by recombinant DNA technology.

The recombinant human G-CSF synthesized in an E. coli expression system is called filgrastim. The structure of filgrastim differs slightly from the natural glycoprotein. Most published studies have used filgrastim and it was the first form of G-CSF to be approved for marketing in Australia.

Another form of recombinant human G-CSF called lenograstim is synthesized in Chinese hamster ovary (CHO) cells. As this is a mammalian cell expression system, lenograstim is indistinguishable from the 174 amino acid natural human G-CSF. No clinical or therapeutic consequences of the differences between filgrastim and lenograstim have yet been identified, but there are no formal comparative studies. G-CSF should not be confused with granulocyte macrophage colony stimulating factor (GM-CSF), which is a distinctly different hematopoietic growth factor also under clinical development.

G-CSF (filgrastim) is indicated for the prevention of febrile neutropenia in patients receiving myelosuppressive chemotherapy for non-myeloid malignancies. It reduces the duration and severity of post-chemotherapy neutropenia.

G-CSF (lenograstim) is also approved for use to reduce the incidence of infection associated with established cytotoxic chemotherapy.
Granulocyte-macrophage colony-stimulating factor: An acidic glycoprotein of mw 23 kD with internal disulfide bonds. It is produced in response to a number of inflammatory mediators by mesenchymal cells present in the hematopoietic environment and at peripheral sites of inflammation. It stimulates the production of neutrophilic granulocytes, macrophages, and mixed granulocyte-macrophage colonies from bone marrow cells and can stimulate the formation of eosinophil colonies from fetal liver progenitor cells. It also has some functional activities in mature granulocytes and macrophages. It is used to promote the recovery of the white blood cells following chemotherapy.
Interleukin-6: A cytokine that stimulates the growth and differentiation of human B-cells and is also a growth factor for hybridomas and plasmacytomas. Many different cells including T-cells, monocytes, and fibroblasts produce it. A single chain 25 kD cytokine originally described as a pre B-cell growth factor, now known to have effects on a number of other cells including T-cells that are also stimulated to proliferate. It induces acute phase proteins and colony-stimulating factor acting on mouse bone marrow.
Cytokine: Small proteins or biological factors (in the range of 5-20 kD) that are released by cells and have specific effects on cell-cell interaction, communication and behavior of other cells. Not really different from hormones, but the term tends to be used as a convenient generic shorthand for interleukins, lymphokines and several related signaling molecules such as TNF and interferons. Generally growth factors would not be classified as cytokines, though TGF is an exception. Natively glycosylated mammalian biological molecules such as G-CSF, GM-CSF, Il-6, Il-8 are extensively used in research and therapeutic treatment. Heretofore, it has been difficult or very expensive to produce these molecules for research or therapeutic use. For instance, while G-CSF is widely used to reduce the duration and severity of post-chemotherapy neutropenia and to induce the survival, proliferation, and differentiation of neutrophilic granulocyte precursor cells and to functionally activate mature blood neutrophils in transplant procedures, and while it is naturally produced in the human body, the isolation of human G-CSF has not been commercially achieved. Consequently, the production of G-CSF has been commercially accomplished only by “synthetic” means such as recombinant DNA technology producing G-CSF synthesized in an E. coli expression system or recombinant human G-CSF synthesized in Chinese hamster ovary (CHO) cells. Both of these “synthetic” processes are costly making the product achieved thereby expensive and thereby creating an additional burden to the already over-burdened health care system.

There are extensive publications on techniques to increase natively glycosylated mammalian biological molecules in humans and laboratory animals and the therapeutic effect derived there from. However, like the problem associated with obtaining commercial quantities of reasonably priced G-CSF, the obtaining of reasonably priced quantities of GM-CSF, cytokines, interleukins, and other desired natively glycosylated mammalian biological molecules has not been accomplished.

The present invention overcomes the problems of prior processes and systems and provides an economical system of producing commercial quantities of natively glycosylated mammalian biological molecules.

### SUMMARY OF THE INVENTION

The present invention relates to a process for producing natively glycosylated mammalian biological molecules, such as mammalian cells, human cells, within a culture medium. The cells are preferably exposed to an electromagnetic field, which, in the preferred embodiment, is a time-varying electromagnetic field.

The cells are preferably grown in a bioreactor in a manner so that they maintain their three dimensional geometry. In a preferred embodiment, the presence of time varying electromagnetic field potentiates the rapid growth of cells.

The system and process are utilized in combination with tissue culture processes to produce growth of natively glycosylated mammalian biological molecules. In this environment, growth-promoting genes are up regulated and growth inhibitory genes are down regulated. The effect is shown to persist over a period of time after termination of the process. It is an object of the present invention to provide a process for producing natively glycosylated mammalian biological molecules.

Another object of this invention is to provide a composition comprising a mixture of natively glycosylated mammalian biological molecules produced by electromagnetically stimulating living mammalian cells.

Still another object of this invention is to provide a composition comprising a mixture of natively glycosylated mammalian biological molecules including proteins, peptides, polypeptides, glycoproteins, cytokines, post-translational proteins, post-translational peptides, and post-translational polypeptides.

It is still another object of this invention to produce a mixture of natively glycosylated mammalian biological molecules that can be separated into its individual component parts for later research or therapeutic use.

It is a further object of this invention to provide a method of producing natively glycosylated mammalian biological molecules utilizing an electromagnetic force to produce a mixture of natively glycosylated mammalian biological molecules present in a harvestable amount in a liquid, and thereafter separating one or more of the natively glycosylated mammalian biological molecules from the mixture.

It is still another object of this invention to provide a method for producing natively glycosylated mammalian biological molecules in which mammalian cells and a carrier medium are introduced into a chamber capable of sustaining cell growth, maintaining the mammalian cells and carrier medium in the chamber under cell growing conditions until natively glycosylated mammalian biological molecules are present in a harvestable amount in the carrier liquid, and separating one or more of the natively glycosylated mammalian biological molecules from the carrier medium. It is a more specific object of this invention to provide a process for producing natively glycosylated mammalian biological molecules in which the natively glycosylated mammalian biological molecules are a member selected from the group comprising proteins, peptides, polypeptides, glycoproteins, cytokines, post-translational proteins, post-translational peptides, and post-translational polypeptides, including specifically G-CSF, G-MCSF, and the interleukins, and where a time varying electromagnetic force is utilized to effect the production.

Other aspects, features and advantages of the present invention will be apparent from the following description of the presently preferred embodiments of the invention given for the purpose of disclosure.

### BRIEF DESCRIPTION OF THE DRAWINGS

FIG. 1 is a side view of the bioreactor used in the present invention;

FIG. 2 is a perspective view of the bioreactor used in the present invention; and

FIG. 3 is an exploded view of the bioreactor used in the present invention.

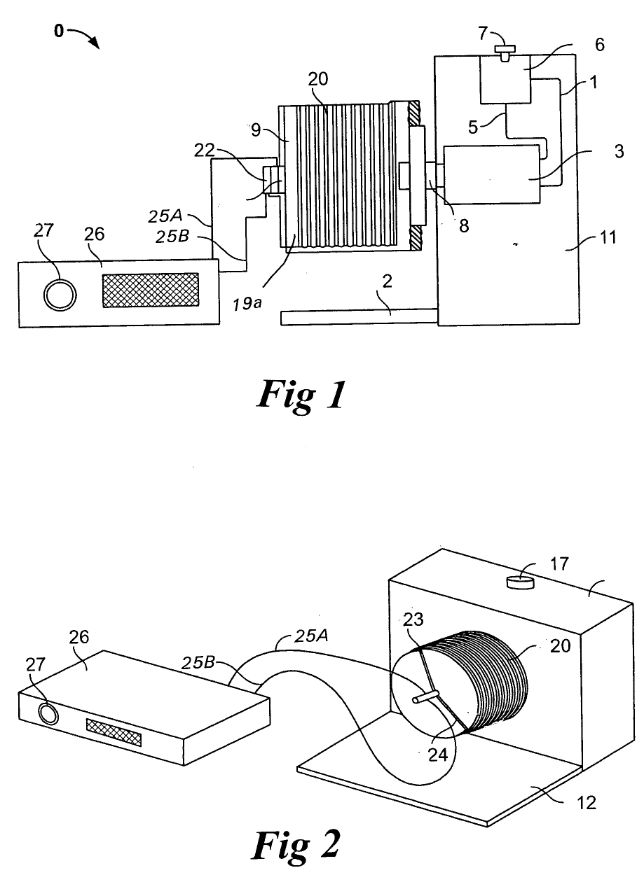

In FIGS. 1 and 2 of the drawings a motor housing 11 is supported by a base 12. A motor 13 is attached inside the motor housing 11 and connected by wires 14 and 15 to a control box 16 that has a control mechanism therein such that the speed of the motor can be incrementally controlled by turning the control knob 17. The motor housing 11 has a motor 13 inside so that the motor shaft 18 extends through the housing with the motor shaft 18 being longitudinal, that is, so that the center of the shaft is parallel to the plane of the earth at the location of the bioreactor 10. A longitudinal cylinder 19 is connected to the shaft so that the cylinder rotates about its longitudinal axis with the longitudinal axis parallel to the plane of the earth. The cylinder is wound on its outside wall 19a with a wire coil or conductor 20. The size of the wire and number of times it is wound around the cylinder are such that when a square wave current of from 0.1 mA to 1000 MA is supplied to the wire coil, an electromagnetic field of from 0.05 gauss to 6 gauss is generated within the cylinder. The wire coil 20 is connected to rings 21 and 22 at the end of the shaft by wires 23 and 24. These rings are then contacted by wires 25A and 25B in such a manner that the cylinder can rotate while the current is constantly supplied to the coil. An electromagnetic generating device 26 is connected to the wires 25A and 25B. The electromagnetic generating device supplies a square wave to the wires and coil by adjusting its output by turning the knob 27.

In operation, the cylinder is opened and the cell culture and carrier liquid placed therein. The cells are obtained from readily available sources. The rotation of the cylinder is adjusted visually so that the cell culture substantially remains at or about the longitudinal axis of the cylinder. The electromagnetic generating device is activated and adjusted so that the square wave output generates the desired electromagnetic field in the cylinder, from 0.05 gauss to 6 gauss. The electromagnetic field can be determined by the number of windings of the coils and the current used by using the formula for Fourier curves (square waves).

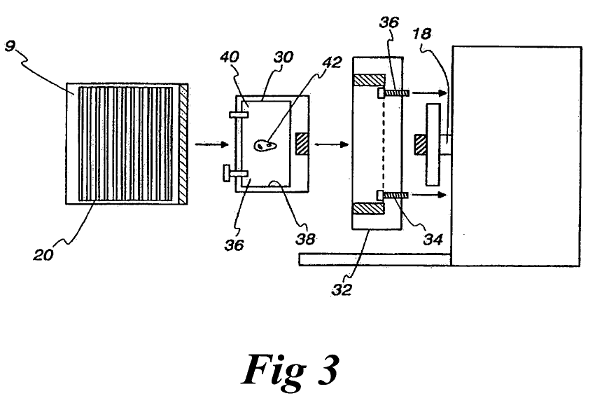

FIG. 3 shows a partial section and exploded view of bioreactor 10. Culture container or rotating wall vessel 30 is shown removed from mounting 32. Bolts 34 attach the container 30 and cylinder 19. The inside area 36 of container 30 with inside wall 38 is filled to near capacity with the liquid all growth media 40 which takes about 95% to 98% of the volume inside area 36 of container 30. In operation, representative cells 42 are suspended in the liquid growth media 40 as the vessel is rotated. The rotations may be from about 2 to about 30 rpm. In order for the cells 42 to stay in the center of fluid filled container 30 and do not touch the inside wall 38 of container 30 they must be visually monitored. When the cells increase in viscosity they may gravitate towards the wall of the vessel. To maintain their position in the center of the fluid filled vessel the rotational speed may be decreased. Thus, the cells cannot be damaged and are allowed to grow or expand at a significant rate, about 7 to 10 times their original size as obtained from peripheral blood. The placement of the cells 42 in the center of liquid filled container 30 can be monitored visually by observing the location of the cells 42 upon rotation because vessel 19 which encloses container 30, are made of a clear plastic material. The inclusion of cells and liquid media in the container 30, nor the presence of wire coil or conductive material 20, does not obscure the viewing of cells 42 by an observer. After a period of time, the rotation is stopped, the container opened, and the mixture therein separated. The cells are discarded and the supernatant liquid is separated into its component parts.

### DETAILED DESCRIPTION OF THE INVENTION

This invention may be more fully described by the preferred embodiment as hereinafter described.

The preferred embodiment of this invention produces a mixture of natively glycosylated mammalian biological molecules produced by electromagnetically stimulating living mammalian cells. Preferably, the natively glycosylated mammalian biological molecules are produced in three-dimensional conditions and the electromagnetic stimulation is provided by applying a time varying electromagnetic force, and, more specifically, a square wave. It is preferred that the natively glycosylated mammalian biological molecules are a member selected from the group comprising proteins, peptides, polypeptides, glycoproteins, cytokines, post-translational proteins, post-translational peptides, and post-translational polypeptides, including specifically G-CSF, GM-CSF, Interleukin-6, and Interleukin-8.

The stated mixture is a mixture found in a supernatant liquid produced by mixing a cell culture and a carrier medium together and subjecting it to the electromagnetic force until the supernatant liquid has a harvestable amount of the natively glycosylated mammalian biological molecules. The supernatant liquid is then separated into its component parts for research or therapeutic use. The mammalian biological material being stimulated is preferably human cells, such as progenitor cells or neuronal cells.

The preferred method for producing the natively glycosylated mammalian biological molecules comprises: (a) introducing mammalian cells and a carrier medium into a cylindrical chamber; (b) rotating the cylindrical chamber about its axis at a rotational speed sufficient to prevent the cells from substantially contacting the cylindrical walls of the cylindrical chamber; (c) continuing the rotation until natively glycosylated mammalian biological molecules are present in a harvestable amount in the carrier liquid; and (d) separating one or more of the natively glycosylated mammalian biological molecules from the carrier medium. This invention also includes a method of therapeutically treating mammals comprising producing the natively glycosylated mammalian biological molecules as described herein and thereafter administering a therapeutical amount of the natively glycosylated mammalian biological molecules to a mammal to achieve a therapeutical affect.

In a preferred embodiment of the invention, normal human neuronal progenitor cells (NHNP) were pooled from three donors to diminish donor-to-donor variations in response. As controls, NHNP were grown in conventional tissue culture following standard cell culturing procedures in tissue culture flasks obtained through Clonetics Corporation, San Diego, Calif.

### Cell Culture Protocols:

For two-dimensional culture, GTSF-2 medium with 10% FBS, Ciprofloxacin and Fungizone was used to culture the cells (Goodwin et al., 1993a). 1×PBS, Collagenase, DNase and trypsin were purchased from Clonetics San Diego, Calif., and used Coming T-75 flasks (Coming Inc., Corning, N.Y.) for initial cell culture to obtain the appropriate number of cells for each experiment. Briefly, cells to be cultured were enzymatically dissociated with the referenced reagents from T-flasks, washed once with PBS-CMF and assayed for viability by trypan dye exclusion (GIBCO, Grand Island, N.Y.). Cells were grown on 100-mm petri dishes (tissue culture treated to prevent adherence) or grown on the actual electrodes inside the petri dishes. Electrodes were made of platinum and stainless steel. Cell cultures were maintained in a humidified Forma CO₂ incubator (Forma, Inc.) at 37° C. at a CO₂ concentration of 6%.

For three-dimensional culture, NHNP cells were prepared as described above and an RWV was sequentially inoculated with 5 mg/ml Cytodex-3 type I collagen-coated microcarriers (Pharmacia) and freshly digested NHNP cells, yielding a cell density of 2.5.×10₅ cells/ml in a 55-ml vessel. Tissues were cultured for 17 to 21 days or until 3- to 5-mm diameter tissue masses formed.

### Generator:

A waveform (TVEMF) generator of original design and capability was developed and used to generate the waveform in a strength of 1-6 mA (AC) square wave, 10 Hz variable duty cycle, which was pulse-width modulated as described in the description of the drawings above. NHNP cells were subjected to these extremely low-level magnetic fields (ELF waves) (˜10-200 mGauss), which are far less than the field strength of the Earth.

### Two-Dimensional Experimental Protocols:

Initially, a metal electrode was placed inside a petri dish and centered. NHNP were seeded at 2.5·105 cells in 0.7 ml of media and carefully dropped on the electrode in a concentrated bubble. Cells were incubated for 2 days. The second day after cell inoculation is considered day 0 of the experiment protocol. At day 0, each dish was given 15 ml of media and waveform was applied to the electrodes. Cells were fed with 15 ml of media at day 3 and with 13 ml every three days thereafter at day 6, 9, and 12. At days 14 and 17, the cells were fed again with 15 ml of media. At days 17 to 21, the cells were incubated for 10 minutes in a Collagenase/DNase cocktail, then trypsin was directly applied to the cocktail and the cells were further incubated for 3 more minutes. Before the complete media was added to deactivate the trypsin, the cocktail mix was pipetted up and down several times. The cells were washed twice with 1×PBS, reapplied with the media, and placed on ice. The cells were observed under a dissecting microscope, counted, and assessed for viability.

An identical protocol was followed in similar experiments with the exception that, instead of the electrode being placed within the petri dishes, in media, it was attached to the underside of the TVEMF treated dishes, so that the cells had no direct contact with the metal surface.

### Three-Dimensional (RWV) Experimental Protocol:

Three-dimensional neural cells and tissues were cultured by the method described above, except that the TVEMF RWV was modified to incorporate an electromagnetic coil. The coil was wrapped around the core of the vessel so it emitted the same electromagnetic field strength as in the two-dimensional configuration. All other conditions were identical to the two-dimensional experimental conditions.

The supernatant liquid was removed from the mixture and analyzed. (describe analysis).

The analysis provided the following results. (Insert Excel spreadsheets).

Lengthy table referenced here
US20090081751A1-20090326-T00001
Please refer to the end of the specification for access instructions.

Lengthy table referenced here
US20090081751A1-20090326-T00002
Please refer to the end of the specification for access instructions.

The results clearly show that this invention provides a new and unique mixture of natively glycosylated mammalian biological molecules that can be separated into therapeutic amounts of highly desirable natively glycosylated mammalian biological molecules including growth factors.

LENGTHY TABLES
The patent application contains a lengthy table section. A copy of the table is available in electronic form from the USPTO web site (http://seqdata.uspto.gov/?pageRequest=docDetail&DocID=US20090081751A1). An electronic copy of the table will also be available from the USPTO upon request and payment of the fee set forth in 37 CFR 1.19(b)(3).

1. A method for producing natively glycosylated mammalian biological molecules: comprising: (a) introducing mammalian cells and a carrier medium into a cylindrical chamber; (b) rotating the cylindrical chamber about its axis at a rotational speed sufficient to prevent the cells from substantially contacting the cylindrical walls of the cylindrical chamber; (c) continuing the rotation until natively glycosylated mammalian biological molecules are present in a harvestable amount in the carrier liquid; and (d) separating one or more of the natively glycosylated mammalian biological molecules from the carrier medium.

2. The method of claim 1 wherein the natively glycosylated mammalian biological molecules are natively glycosylated human molecules.

3. The method of claim 1 wherein the natively glycosylated mammalian biological molecules are a member selected from the group comprising proteins, peptides, polypeptides, glycoproteins, cytokines, post-translational proteins, post-translational peptides, and post-translational polypeptides.

4. The method of claim 1 wherein the natively glycosylated mammalian biological molecules are a member selected from the group comprising human proteins, human peptides, human polypeptides, human glycoproteins, human cytokines, human post-translational proteins, human post-translational peptides, and human post-translational polypeptides.

5. The method of claim 1 wherein the mammalian cells that are introduced into the cylindrical chamber with the carrier medium are human cells.

6. The method of claim 5 wherein the human cells are progenitor cells.

7. The method of claim 6 wherein the progenitor cells are neural progenitor cells.

8. The method of claim 5 wherein the natively glycosylated mammalian biological molecules are a member selected from the group comprising granulocyte colony stimulating factor, granulocyte macrophage colony stimulating factor, and interleukin-6.

9. The method of claim 1 wherein an electromagnetic force is applied to the cylindrical chamber as it rotates.

10. The method of claim 9 wherein the electromagnetic force is a time varying electromagnetic force.

11. The method of claim 10 wherein the time varying electromagnetic force is in the form of a square wave.

12. The method of claim 1 wherein the cylindrical chamber is selected from the group consisting of a rotating perfused vessel and a rotating wall batch-fed vessel.

13. A method for producing natively glycosylated mammalian biological molecules comprising: (a) introducing mammalian cells and a carrier medium into a chamber capable of sustaining cell growth; (b) maintaining the mammalian cells and a carrier medium in the chamber under cell growing conditions until natively glycosylated mammalian biological molecules are present in a harvestable amount in the carrier liquid; and (c) separating one or more of the natively glycosylated mammalian biological molecules from the carrier medium.

14. The method of claim 13 wherein the natively glycosylated mammalian biological molecules are natively glycosylated human molecules.

15. The method of claim 13 wherein the natively glycosylated memmalian biological molecules are a member selected from the group comprising proteins, peptides, polypeptides, glycoprotiens, bytokines, post-tranlational proteins, post-tranlsational peptides, and post-tranlational polypeptides.

16. The method of claim 15 wherein the natively glycosylated mammalian biological molecules are a member selected from the group comprising human proteins, human peptides, human polypeptides, human glycoproteins, human cytokines, human post-translational proteins, human post-translational peptides, and human post-translational polypeptides.

17. The method of claim 13 wherein the mammalian cells that are introduced into the cylindrical chamber with the carrier medium are human cells.

18. The method of claim 17 wherein the human cells are progenitor cells.

19. The method of claim 18 wherein the progenitor cells are neural progenitor cells.

20. The method of claim 13 wherein the natively glycosylated mammalian biological molecules are a member selected from the group comprising granulocyte colony stimulating factor, granulocyte macrophage colony simulating factor, and interleukin-6.

21. The method of claim 13 wherein an electromagnetic force is applied to the chamber to induce the material therein to proliferate.

22. The method of claim 21 wherein the electromagnetic force is a time varying electromagnetic force.

23. The method of claim 22 wherein the time varying electromagnetic force is in the form of a square wave.

24. The method of claim 13 wherein the chamber is selected from the group consisting of a rotating perfused vessel and a rotating wall batch-fed vessel.
